(19) United States
(12) Patent Application Publication
(10) Pub. No.: US 2003/0105462 A1
(43) Pub. Date: Jun. 5, 2003

(54) Poly axial cervical plate system

(76) Inventor: Thomas T. Haider, Riverside, CA (US)

Correspondence Address: Harold L. Jackson, Jackson Law Corporation, Ste. N, 14751 Plaza Dr., Tustin, CA, 92780, US

(21) Appl. No.: 10/020,035

(22) Filed: Nov. 30, 2001

(51) Int. Cl. A61B 17/56

(52) U.S. Cl. 606/69

(57) ABSTRACT

A poly axial cervical plate system for immobilizing bone segments for use with a wrench comprising a screw that has a plurality of spring element on the head of the screw and a plate having a number of screw receiving holes. The wrench compresses the spring elements into a contracted position such that the head of the screw is rotatable within the screw receiving hole while the wrench is used to secure the screw into the bone. When the wrench is removed from the head of the screw, the spring elements extend. The peripheral surface of the head of the screw in the extended position is substantially convex and the surface of the screw receiving hole is substantially concave. The head of the screw in the extended position contacts the surface of the screw receiving hole such that the screw is substantially secured in place.

### BACKGROUND OF THE INVENTION

1. Field of the Invention

This invention relates to a poly axial cervical plate system for use in immobilizing bone segments.

2. Description of the Prior Art

Damage to individual bones or bone segments can occur from such things as disease and physical injury. When damage does happen it is often necessary to immobilize the bone or bones to promote healing. This occurs, for example, when attempting to achieve osteosynthesis or fusion between elements of a bone or bone segments.

In such cases, it is imperative that the bone elements to be fused must be kept in close proximity and relatively fixed in place to allow the bone to grow and harden. Furthermore, it is important to use as little hardware as possible since, in instances such as when spinal bone segments are concerned, the working area can be very small. Also, time may be critical element considering that a patient is usually under anesthesia during placement of the hardware.

In areas such as the spine the desired pattern of bone immobilization and resulting bone fusion usually follows a shape that is generally along the shape of the spinal column. In such instances, it is necessary to have equipment that is versatile and accommodates the desired shape and placement for a patient, yet when installed will provide a relatively rigid fixation of the bone or bone segments.

The rigid fixation necessary in healing or fusing bones requires that the hardware utilized must not loosen after being installed. However, the devices incorporated must be easy to remove if necessary.

A variety of rigid fixation systems are well known in the field. For example, U.S. Pat. No. 6,139,550 to Michelson (the '550 patent) claims a plate for use with a bone screw and locking element. In the case of passive dynamic and self-compressing systems, the plate allows for a poly axial movement of a screw that has a rounded head. Once installed in the proper angle, the bone screw is secured in place by a locking element.

The '550 patent requires three elements to provide fixation; the plate, screw, and locking element. Again, due to limited working areas and time considerations, a system with fewer elements would be preferable.

U.S. Pat. No. 6,235,034 to Bray (the '034 patent) identifies a screw, base plate, retaining plate and set screw. The base plate and retaining screw are used in conjunction to prevent the screw from backing out of the bone. This appears to be best suited for a passive dynamic, but not a self-compressing system. This system has more parts than the '550 patent, which in turn is less desirable in small areas.

U.S. Pat. No. 6,235,033 to Brace and Hansjuerg (the '033 patent) is another poly axial type bone fixation assembly. The '033 patent uses a screw, locking screw, plate, and bushing. Poly axial rotation of the screw is accomplished through the interface of the bushing and the receiving hole in the plate. The locking screw secures the screw in a specific position by forcing the bushing into contact with the surface of the hole.

This procedure requires alignment of the screw and bushing followed by use of the locking screw. A less complicated device would be desirable.

The present invention offers the advantage of having less component parts while providing the option of being used in passive dynamic or self-compressing applications.

### SUMMARY OF THE INVENTION

The invention is directed to a poly-axial cervical plate system used to promote healing of damaged bones or bone segments. A screw and plate are used in conjunction with a wrench to secure bones or bone segments in place. The screw has a head with a spherical periphery and a plurality of spring segments adapted to receive the wrench. The plate has a plurality of screw receiving holes, each hole having a concave like inside surface. When the wrench engages the spring segments, the segments are brought into a contracted position and the wrench can drive the screw into the bone. In this position, the head of the screw fits within the screw receiving hole of the plate and the screw has a poly axial range of motion. When the wrench is removed, the spring segments expand into an expanded position. In the expanded position, the peripheral surface of the head of the screw comes into contact with the surface of the screw receiving hole. Depending upon the application, the degree of poly axial movement of the head of the screw in this position can be substantially limited ranging from some degree of movement to being firmly secured into place.

### BRIEF DESCRIPTION OF THE DRAWINGS

FIG. 1 is a perspective view of the screw.

FIG. 1a is a top view of the head of the screw with the spring segments in the extended position.

FIG. 1b is a top view of the head of the screw with the spring segments in the contracted position.

FIG. 1c is a side view of the head of the screw.

FIG. 2 is a perspective view of the plate.

FIG. 3 is a cutaway side view of the plate.

FIG. 3a is a cutaway side view of the plate with the screws set in position.

FIG. 4 is a perspective view of the palte with a screw secured in position.

FIG. 5 is a side view of a wrench suitable for inserting the screw.

FIG. 6 is an end view of the engaging end of the wrench.

FIG. 7 is an exploded view of the wrench.

FIG. 8 is a side view of the wrench engaging the screw, the screw secured into the bone, and the plate secured in position.

### DETAILED DESCRIPTION OF THE DRAWINGS

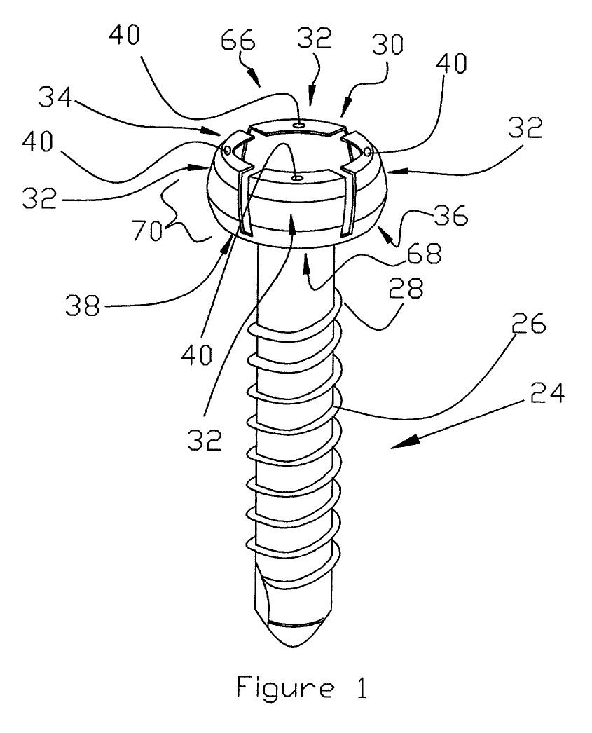

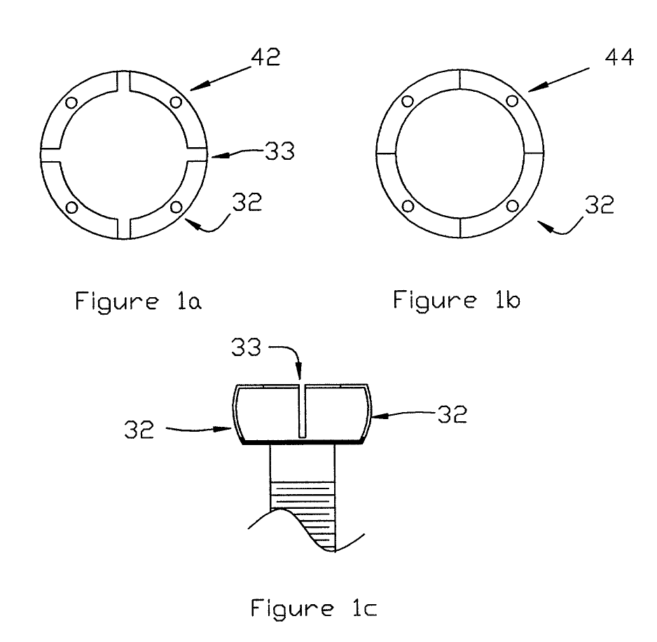

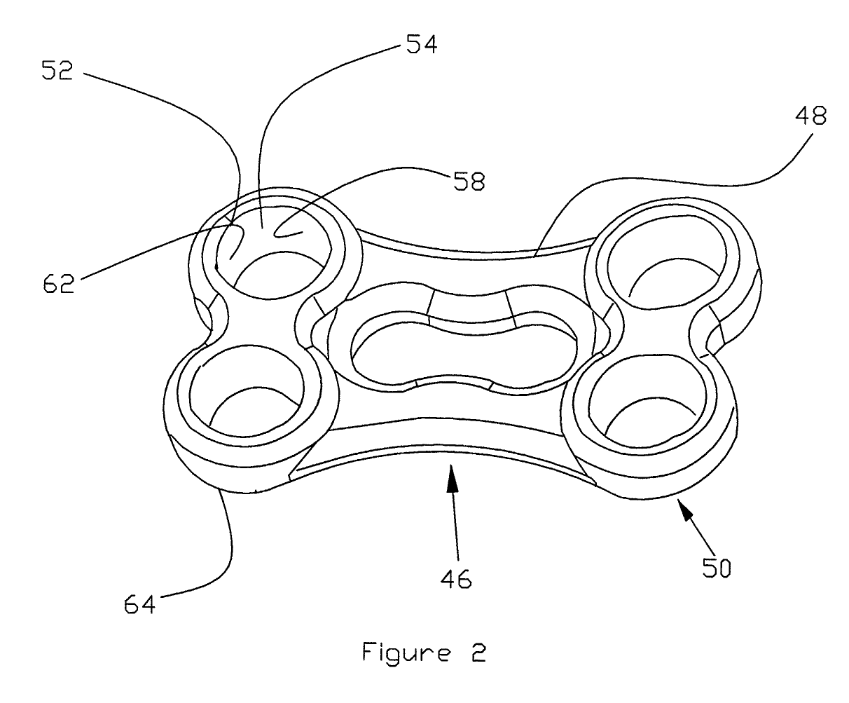

The present invention may best be understood by reference to the following description taken in conjunction with the accompanying drawings. FIG. 1 is a perspective view of the screw 24. The screw 24 is comprised of a head 30 and a cylindrical threaded shaft 26 having a cylindrical diameter 28 (i.e., outside diameter of the threads). The head has a top surface 34, a bottom surface 36, a peripheral side 70, and plurality of spring segments 32 separated by a slot 33. Each of the spring segments 32 has a wrench engaging surface 40, i.e. holes in the top surface 34. It is important to note that while the top surface 34 and bottom surface 36 appear in FIG. 1 as being flat, this is not necessary for the invention. The surfaces can be other than flat, i.e. round.

Turning now to FIG. 1a, when the spring segments 32 are in the extended position 42, the slot 33 form a space between the spring segments 32. In FIG. 1b, the spring segments 32 are in the contracted position 44 and little to no space exists between the spring segments 32 from this top view. FIG. 1c shows a side view of the spring segments 32 and a slot 33 between the spring segments 32.

FIG. 2 is a perspective view of the plate 46. The plate 46 has a top surface 48, a bottom surface 50, (FIG. 2) and a plurality of screw receiving hole 52. Each screw receiving hole 52 has a screw head receiving surface 54 that has a substantially spherical inside surface 60 with a top diameter 62, a maximum intermediate diameter 58, and a bottom diameter 64. the top diameter 62 and the bottom diameter 64 being larger than the cylindrical diameter 28 of the cylindrical threaded shaft 26 thereby accommodating the passage of the cylindrical threaded shaft 26 therethrough.

The top diameter 62 of the screw receiving hole 52 is larger than the maximum diameter of the peripheral side 70 of the head 30 of the screw 24 when it is in the contracted position 44 as shown in FIG. 1b, thereby allowing poly axial movement of the screw 24 within the plate 46. The bottom diameter 64 being smaller than the maximum diameter of the peripheral side 70. This allows the head 30 of the screw 24 in FIG. 1 to fit within the screw receiving hole 52 of FIG. 2 when the head 30 is contracted and yet not to pass through the bottom diameter 64. When the head 30 is in the extended position 42, the peripheral side 70 has a substantially convex surface that engages the substantially spherical inside surface 60 of the screw receiving hole 52 of FIG. 2 in such a way as to secure the head 30 in place.

In cases where it is desirable for the head 30 to have a limited poly axial movement when in the extended position 42, the fit between the substantially spherical surface of the peripheral side 70 and the substantially spherical inside surface 60 of the screw receiving hole 52 can be such that the head 30 is not secured in one position. The head 30 can be substantially secured, which means between a range encompassing the head 30 being firmly secured to where the head 30 has a degree of poly axial movement when in contact with the substantially spherical inside surface 60 of the screw receiving hole 52.

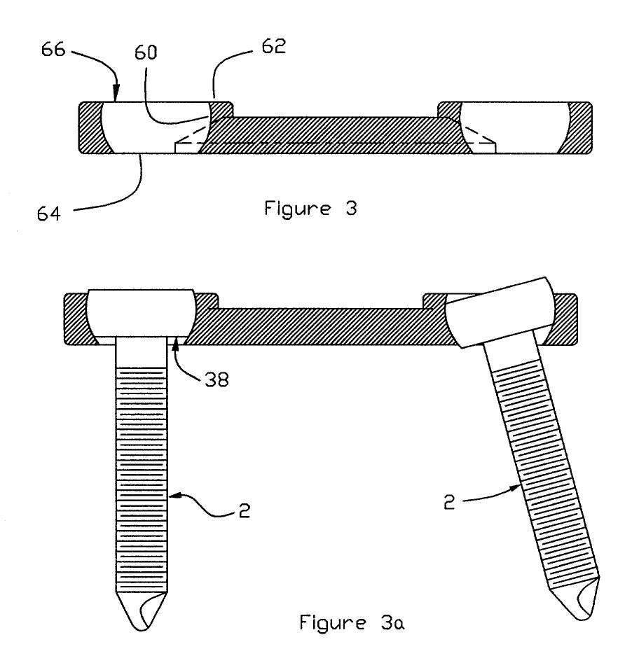

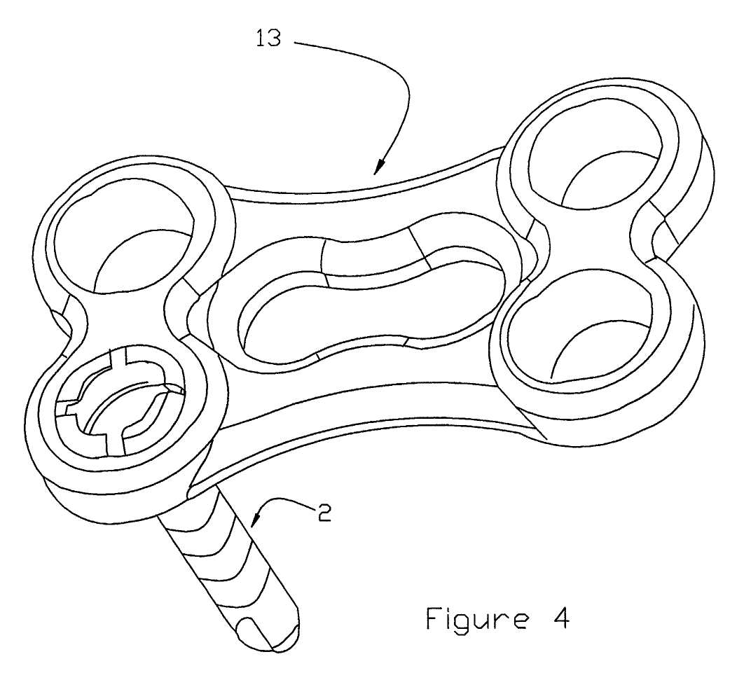

FIG. 3 is a cutaway side view of the plate 46 exhibiting the substantially spherical inside surface 60 of the screw head receiving surface 54. FIG. 3a displays the head 30 of the screw 24 with the peripheral side 70 engaging the substantially spherical inside surface 60 as the head 30 is in the extended position 42 and thus secured in place. When the spring segments 32 are in the contracted position 44 as in FIG. 1b, the screw 24 is free to rotate and exhibits poly axial movement. FIG. 4 is a perspective view of the screw 24 secured in place in the screw receiving hole 52.

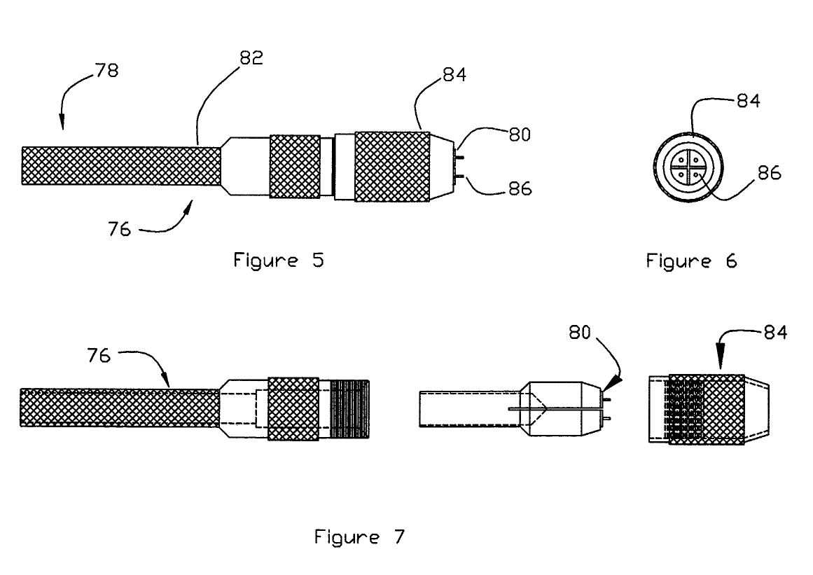

The wrench 76 of FIG. 5 has an elongated cylindrical body 78, a grasping end 82, adjustable collar 84, and a screw engaging end 80 that has projections 86 to engage the wrench engaging surface 40 of the screw 24 in FIG. 1. FIG. 6. is an end view showing the adjustable collar 84 and projections 86. In FIG. 7, the wrench 76 is shown in an exploded view showing the components.

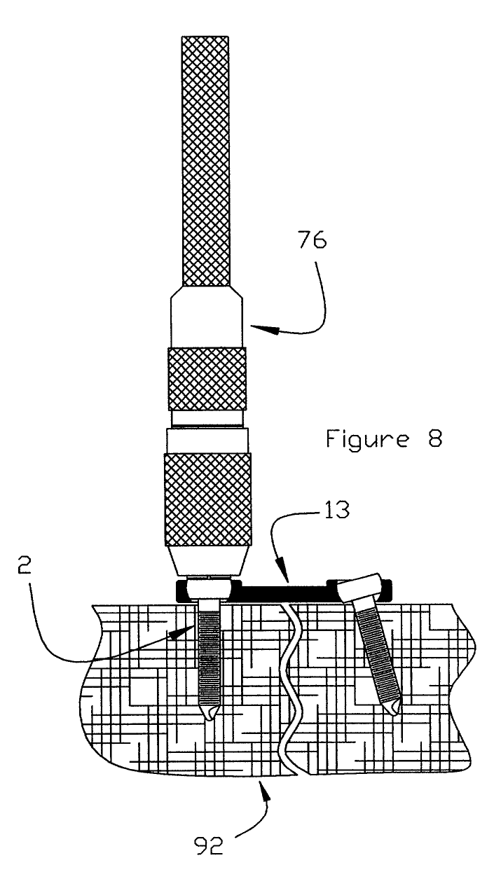

Movement of the adjustable collar 84 translates into movement of the projections 86 and thus the spring segments 32. As the projections 86 move inward in response to movement of the adjustable collar 84 while the projections 86 are within the wrench engaging surface 40, the spring segments 32 go to the contracted position 44. When the projections 86 is adjusted to move the projections 86 outward, the spring segments 32 got to the extended position 42. FIG. 8 shows the wrench 76 engaging the screw 24 and securing the screw 24 into the bone 92. As the screw 24 is rotated within the bone 92, the plate 46 is brought into contact with the bone 92. When the wrench 76 is disengaged, the screw is secured into place within the plate 46 and the bone is thereby brought into place for healing.

What is claimed is:

1. A poly axial cervical plate system for immobilizing bone segments comprising;
a) a screw having a cylindrical threaded shaft and a head, the head defining a plurality of spring segments and a top surface, the bottom of the head being joined to the cylindrical threaded shaft, the top surface defining a wrench engaging surface for each spring segment, the spring segments normally being in an extended position and arranged to converge to a contracted position in response to an inward force being applied to the wrench engaging surface; and
b) a plate having a top and bottom surface and having a plurality of screw receiving holes extending from the top to the bottom surfaces of the plate, each screw receiving hole having a surface being arranged to allow the cylindrical threaded shaft to pass therethrough but not the head and to accommodate the head of the screw therein when the segments are in a contracted position and allowing for poly axial movement of the screw, the surface of the hole engaging the head of the screw being arranged so that the head of the screw is substantially secured in place when the head of the screw is in the extended position.

2. A poly axial cervical plate system for immobilizing bone segments for use with a wrench as in claim 1, wherein the wrench further comprises a plurality of projections and each wrench engaging surface being adapted to receive a projection and the wrench forcing the projections inward so that the head of the screw is in a contracted position and release of the inward force resulting in the head of the screw being in an extended position.

3. A poly axial cervical plate system for immobilizing bone segments for use with a wrench comprising:
a) a screw having a cylindrical threaded shaft defining a cylindrical diameter and a head, the head defining a plurality of spring segments, a top and bottom surface defining a top and bottom diameter and a peripheral side, the bottom diameter being larger than the diameter of the cylindrical threaded shaft and the bottom surface of the head being joined to the cylindrical threaded shaft, the top surface defining a wrench engaging surface for each spring segment, the peripheral side having a substantially convex surface defining a maximum diameter, and the spring segments normally being in an extended position and arranged to contract in response to the wrench engaging the wrench engaging surfaces; and
b) a plate having a top and bottom surface and defining a plurality of screw receiving holes each hole having a concave inside surface substantially matching the convex surface of the screw head and extending from the top to the bottom surfaces of the plate, the inside surface of the holes further defining a top and bottom diameter, the bottom diameter of the hole being larger than the diameter of the cylindrical threaded shaft and smaller than the maximum diameter of the head of the screw in its contracted position the top diameter of the hole being greater than the bottom diameter and adapted to receive the cylindrical threaded shaft and the head of the screw when the head of the screw is in a contracted position, the substantially concave shape of the inside surface of the hole allowing for poly axial movement of the screw, the substantially concave shape of the inside surface of the hole adapted to engage the substantially convex outer periphery of the head of the screw when the screw head is in the extended position.

4. A screw for use with a wrench and a poly axial cervical plate system, the screw comprising:
a cylindrical threaded shaft defining a diameter and a head, the head defining a plurality of spring segments, a top and bottom surface defining a top and bottom diameter and a peripheral side, the bottom diameter being larger than the diameter of the cylindrical threaded shaft and the bottom surface of the head being joined to the cylindrical threaded shaft, the top surface defining a wrench engaging surface for each spring segment, the peripheral side having a substantially convex surface defining a maximum diameter, and the spring segments normally being in an extended position and being in a contracted position when the wrench engages the wrench engaging surface and when the wrench engages the wrench engaging surface the screw may be rotatably inserted into a bone segment.

5. A wrench for use with a screw, the head of the screw having a plurality of spring segments and each spring segment having an extended and contracted positions and each spring segment having a wrench engaging surface, the wrench comprising:
an elongated cylindrical body having a screw engaging end and a grasping end, the screw engaging end defining an adjustable collar being movable along the elongated cylindrical body and a plurality of projections having an open position adapted to engage the wrench engaging surfaces of the head of the screw when the wrench engaging surfaces are in an extended position, and the projections having a closed position causing the wrench engaging surface to be in a contracted position, and movement of the collar changes the position of the projections.

6. The method for immobilizing bone segments utilizing a poly axial plate system having a screw defining a head having a plurality of spring segment and each spring segment having an extended and contracted positions, a plate defining a plurality of screw receiving holes and each screw receiving hole adapted to receive the head of the screw in a contracted position and allowing for poly axial movement of the screw and when the head of the screw is in an extended position each screw receiving hole contacting the head of the screw such that the head of the screw is in a substantially secured position, and a wrench adapted to receive the head of the screw with the wrench transitioning the spring segments between the extended and contracted positions and the wrench being adapted to rotatably insert the screw into a bone, comprising the steps of;
a) drilling a hole into the bone segment;
b) using the wrench to place the spring segments in a contracted position;
c) inserting the screw into the screw receiving hole in the plate;
d) rotating the screw into the bone by using the wrench;
e) tightening the screw into the bone such that the head of the screw is within the screw receiving hole of the plate;
f) removing the wrench from the head of the screw thereby causing the spring segments to be in an extended position and thus substantially secured in place;
g) repeating the steps for the required number of screws.
